(19) United States
(12) Patent Application Publication
(10) Pub. No.: US 2016/0051170 A1
(43) Pub. Date: Feb. 25, 2016

(54) Bloodless Glucose Measuring Device and Method of Use Thereof

(71) Applicant: Gien Chung Lin, Shenzhen (CN)

(72) Inventors: Gien Chung LIN, Shenzhen (CN); Clement JanBye LIN, Rowland Heights, CA (US); Ivana LIN, Rowland Heights, CA (US); Lirong ZHAO, Shenzhen (CN)

(73) Assignee: Gien Chung Lin

(21) Appl. No.: 14/466,206

(22) Filed: Aug. 22, 2014

(51) Int. Cl. A61B 5/145; A61B 5/00; A61B 5/053

(52) U.S. Cl. CPC A61B 5/14532; A61B 5/0537; A61B 5/7278; A61B 5/7445; A61B 5/70; A61B 5/6826; A61B 5/7225; A61B 5/486; A61B 5/7246; A61B 5/7275

(57) ABSTRACT

A bloodless glucose measuring and monitoring device which comprises a main computer and a PCB with a LCD display; a microprocessor; a blood glucose detection circuit connected to the microprocessor which includes a blood glucose detection module; and a memory module, a power module and a timekeeping module connected to the microprocessor. The PCB comprises a body resistance acquisition circuit with a body resistance acquisition module electrically connected to the microprocessor for measuring a body resistance between two points of skin of a human body and communicates with the microprocessor such that the body resistance obtained by the body resistance acquisition module is translated into a blood glucose value through the microprocessor and then the blood glucose value is displayed through the LCD display of the main computer.

### BACKGROUND OF THE PRESENT INVENTION

1. Field of Invention

The present invention relates to a medical device, and more particularly to a bloodless glucose measuring and monitoring device and its method of use.

2. Description of Related Arts

Blood glucose level is a very important index for diabetes and therefore is very concerned by many diabetic patients. In fact, fasting plasma glucose level and 2-hour postprandial blood glucose level are used for diagnosis of diabetes. For fasting plasma glucose level, the normal range is 3.9-6. mmol/L (70-110 mg/dL) and a level of 7.0 mmol/L (126 mg/dL) or above confirms the diagnosis of diabetes. In other words, the standard diagnosis of diabetes is based on the standard that a blood glucose level is 7.0 mmol/L or 126 mg/dL, and that there is a gap between the standard diagnosis of diabetes and the normal range. Some people do not have a normal blood glucose level but their glucose level are not high enough for a diagnosis of diabetes, which is referred to as elevated fasting plasma glucose level (impaired fasting glucose). For 2-hour postprandial blood glucose level, the normal range is 3.9-7.8 mmol/L (70-140 mg/dL) and a level of 11.1 mmol/L or 200 mg/dL or above confirms the diagnosis of diabetes. This also shows that there is also a difference between 140 mg/dL and 200 mg/dL, and that a glucose level within this range is referred to as elevated 2-hour plasma glucose. People who have elevated level are neither diagnosis as diabetic, nor normal. They are at high risk to develop diabetes and must be very careful to monitor their blood glucose level. If they do not pay special attention to their situations, they will develop diabetes easily in a short period of time. In addition, people who have normal blood glucose level with high-risk factors of diabetes also need to pay special attention to their blood glucose level. High blood glucose is one of the ‘high’ in “three-high disorder” (which refers to high blood pressure, high blood glucose and high blood cholesterol) and therefore it is very important to monitor the blood glucose level at al times.

At present, most of the blood glucose meters in the market requires blood sampling for testing and blood test requires the use of blood glucose test strip which is complementary to the particular glucose meter, that the test strip is not universal for use in different meters or meters from different brands. At present, there are two types of test strips which require two different blood sampling methods: the blood dropping method and the siphoning method. For test strips which utilize the blood dropping method for sampling, a greater amount of blood sample is required for testing, that the testing involves dropping a blood droplet onto the test strip and the test result will be affected if excessive or inadequate amount of blood sample is applied, or if the position of blood sample is not accurate. For test strips which utilize blood siphoning method for sampling, the test result is accurate but the cost is high, while the use cycle is small and the environmental impact is great.

In addition, most of the glucose meters have the following drawbacks:

1. The accuracy is not sufficiently high. At present, the standard deviation is plus or minus 20% for values greater than 4.2 mmol/L for international standard of glucose meters. In other words, the error in the ranges of plus or minus 20% is acceptable under the international standard. The new international standard will have more stringent requirements, that the standard deviation is plus or minus 15% for values greater than 5.6 mmol/L. The accuracy of hospital instruments is deemed to be 100%.

2. The results are susceptible to interference easily. Due to the limitation posed by reaction enzyme which is used in conventional glucose meters, the presence of object interference such as oxygen or iodine will affect the accuracy of the results. This kind of interference is less significant in hospital instruments.

3. The results of glucose meters are affected by test conditions such as temperature, humidity, altitude and etc. The hospital instruments work under a stable condition and is not affected.

4. The results are affected by human factors. For example, the existence of contaminants such as alcohol or the presence of tissues in blood samples from blood sampling.

Glucose meters are used for measuring glucose level. Unless the glucose test is carried out in hospital, the results from glucose test at home will be affected by the test environment, the knowledge of a user and the familiarly to operation of the user, therefore the error of test results will have a great level of derivation. Accordingly, there is a need to develop a glucose monitoring device which does not require blood sampling and is capable of providing a real-time assessment of blood glucose level such that a user or a diabetic patient can measure his or her blood glucose level easily and conveniently and therefore make appropriate adjustment to his or her diet and lifestyle.

### SUMMARY OF THE PRESENT INVENTION

An object of the present invention is to provide a bloodless glucose measuring device and method of use thereof, that this method utilizes skin resistance which will react in response to changes in human blood glucose level for achieving real-time monitoring of blood glucose level.

In order to achieve the above object, the followings are employed:

A bloodless glucose measuring and monitoring device, which comprises: a main computer and a PCB provided in the main computer, wherein the PCB comprises: a LCD display; a microprocessor; a blood glucose detection circuit connected to the microprocessor which comprises a blood glucose detection module; and a memory module, a power module and a timekeeping module connected to the microprocessor, wherein the PCB comprises a body resistance acquisition circuit electrically connected to the microprocessor, wherein the body resistance acquisition circuit comprises a body resistance acquisition module which detects a body resistance between two points of skin of a human body and communicates with the microprocessor such that the body resistance obtained by the body resistance acquisition module is translated into a blood glucose value through the microprocessor and then the blood glucose value is displayed through the LCD display of the main computer.

In particular, the main computer comprises a main casing defining a bottom casing member; at least two positioning groves for guiding a position of a human finger; and a touch type metal electrode provided on a front end of each the positioning groove connecting to an anode of the body resistance acquisition circuit.

In particular, the device comprises a connecting wire (6) having a first end and a second end; and a handheld electrode (6) having a metal electrode portion peripherally provided on an exterior surface of the handheld electrode (6), wherein the first end of the connecting wire is connected to a cathode of the body resistance acquisition circuit and extending from a side portion of the main casing and the second end of the connecting wire is connected to the metal electrode portion of the handheld electrode (6).

In particular, the device comprises three the positioning grooves defining an index finger groove, a middle finger groove and a ring finger groove arranged for placing a bare index finger, a bare middle finger and a bare ring finger of a human body onto the three positioning grooves in such a manner that each of the bare index, middle and ring fingers of a left hand of the human body are having a direct contact with the touch type metal electrode inside the corresponding positioning groove through pressing against the corresponding positioning grooves while a bare right hand of the human body is in direct contact with the metal electrode portion (61) through holding the handheld electrode (6), then a loop resistance is formed.

In particular, the device comprises a dual operational amplifier connecting to the body resistance acquisition circuit.

The present invention also provide a method of use of the bloodless glucose monitoring device, which comprises the following steps:

(a) selecting a plurality of locations of the human body, each of which is capable of reflecting a blood glucose level of the human body of which the blood glucose level is closely correlated with a body resistance of the particular location of the human body, wherein a change in body resistance of the particular location of the human body is directly correlated to a change in blood glucose level of the particular location of the human body and an amplitude of the change in blood glucose level and an amplitude of the change in body resistance resulted from the change in blood glucose level are the same;

(b) conducting a blood glucose test with blood sample for obtaining a value of blood glucose level which is define as ‘blood glucose level a’ through the main computer, wherein the main computer has a test strip slot provided at an upper portion of the main computer and the blood glucose test is conducted through blood sampling by a test strip and inserting the test strip into the test strip slot to start the bloodless glucose monitoring device and to obtain the blood glucose level a through the bloodless glucose monitoring device blood;

(c) obtaining an average value of body resistance b through measuring a value of body resistance between two arms through the positioning grooves and the handheld electrode repeatedly for a predetermined number of times, therefore a correlation between the blood glucose level and the resistance under a particular body condition is obtained and defined as t=a/b;

(d) after the correlation t=a/b is established, carrying out a bloodless blood glucose test by obtaining a blood glucose level ‘an’ through measuring a body resistance ‘cn’ of a particular body location and utilizing the correlation t for calculation, where an=cn*t, wherein the body resistance measured by the body resistance acquisition module is automatically processed through the microprocessor to translate into a blood glucose level, which is displayed through the LCD display (2);

(e) determining a value of glucose level standard under a particular time condition since a value of glucose level at different time will be different, through conducting a blood glucose test with blood sample through the main computer under a healthy body condition of the human body, measuring the value of glucose level standard which is defined as standard glucose value f, where f is obtained through conducting the blood glucose test with blood sample and is used as a standard for subsequent bloodless glucose test to observe the changes in blood glucose level;

(f) subsequently obtaining a blood glucose value an1 under the particular time condition in the step (e) by carrying out the bloodless blood glucose test with the bloodless glucose monitoring device; comparing the blood glucose value an1 and the standard glucose value f to obtain an amplitude of change in blood glucose level such that the user can be reminded of the changes on a daily basis and react to the changes on a timely basis; and

(g) saving the blood glucose value for each of the bloodless blood glucose test carried out through the bloodless glucose monitoring device by the memory module such that the user can take appropriate measures in response to the changes in blood glucose level.

Furthermore, in the step (a) of the method of use of the device, the locations of the human body has direct effect on the accuracy of the blood glucose value measured by the bloodless glucose monitoring device, wherein the locations being selected has relatively thin skin layer and is in close proximity to a vein of the human body, wherein a blood glucose content in the vein has direct effect on a chemical composition of the skin which is in close proximity to the vein while a skin resistance is closely correlated to the chemical composition of the skin such that a blood glucose change has direct effect on the skin resistance at the locations being selected.

Furthermore, in the step (d) of the method of use of the device, the body resistance measured by the body resistance acquisition module comprises a measurement process comprising two steps, wherein the first step refers to verifying a Vcc voltage through resistance sampling for 34 times, removing the smallest value and the greatest value to obtain an average value AD through formula (a):

$$Vcc = \frac{\frac{AD ◯ 1}{256} \times 2.5 \times ( 510 k + 510 k )}{510 k} = \frac{AD / D \times 5}{256}$$

wherein the second step refers to measuring a body resistance through resistance sampling for every 4 ms for 1000 times, where a total time used is 4 s and an average AD value is obtained through formula (b):

$$\frac{Vcc \cdot \frac{10 k}{10 k + {R}_{body}} \times 10}{2.5} \times 256 = {AD}_{◯ 2}$$

thereby the body resistance of human body is obtained through formula (c):

$${R}_{body} = \frac{1024 \times Vcc}{{AD}_{◯ 2}} - 10 k ;$$

where R_{body} refers to the body resistance being measured.

The present invention has the following advantages:

1. Body blood glucose changes is reflected through measuring skin resistance, therefore blood sampling for each blood glucose test, which is required for traditional method, is eliminated. The user's pain level is reduced while the blood glucose level changes can be determined on a timely basis.

2. The body blood glucose level is determined through body resistance while no blood sampling is required and the process is quick, therefore the users can measure their blood glucose level by themselves at any time, which is convenience and practical to the users.

3. The bloodless glucose monitoring device of the present invention only requires blood sampling for setting up a glucose standard value at the first use and no blood sampling is required subsequently after the glucose standard value is obtained. During the method of use of the bloodless glucose monitoring device of the present invention, the body resistance between two limbs of a body or any two points of a body is measured and then the body resistance value is translated into a blood glucose level value to provide the blood glucose level value onto the LCD display.

4. The bloodless glucose monitoring device of the present invention is easy to use and convenience to operate, therefore providing a great relief to diabetic patients while providing a great monitoring tools for people with normal or impaired glucose level serving as preventive measures to diabetes.

### BRIEF DESCRIPTION OF THE DRAWINGS

FIG. 1 is a front view illustration of the bloodless glucose measuring and monitoring device according to the preferred embodiment of the present invention.

FIG. 2 is a rear view illustration of the bloodless glucose measuring and monitoring device according to the above preferred embodiment of the present invention.

FIG. 3 is an enlarged view illustration of a connecting wire connecting to an anode of a handheld electrode of the bloodless glucose measuring and monitoring device according to the above preferred embodiment of the present invention.

FIG. 4 is an illustration of an alternative arrangement for the anode provided on a bottom surface of a bottom casing member of a main computer of the bloodless glucose measuring and monitoring device according to the above preferred embodiment of the present invention.

FIG. 5 is a flow-chart illustration of a process of blood glucose test with the bloodless glucose measuring and monitoring device according to the above preferred embodiment of the present invention.

FIG. 6 is a schematic diagram of the bloodless glucose measuring and monitoring device according to the above preferred embodiment of the present invention.

FIG. 7 is a circuit diagram of a body resistance acquisition circuit of the bloodless glucose measuring and monitoring device according to the above preferred embodiment of the present invention.

FIG. 8 is a circuit diagram of a display module of the LCD display of the bloodless glucose measuring and monitoring device according to the above preferred embodiment of the present invention.

FIG. 9 is a circuit diagram of a timekeeping module of the bloodless glucose measuring and monitoring device according to the above preferred embodiment of the present invention.

FIG. 10 is a flow diagram for a body resistance acquisition process by the bloodless glucose measuring and monitoring device according to the above preferred embodiment of the present invention.

FIG. 11 is a circuit diagram of a microprocessor of the bloodless glucose measuring and monitoring device according to the above preferred embodiment of the present invention.

Numeral references in the drawings: main computer: 1; LCD display: 2; control button: 3; on/off switch: 4; test strip slot: 5; handheld electrode: 6; connecting wire: 7; positioning groove: 11; touch type metal electrode: 12; touch type metal electrode 13; metal electrode portion 61.

### DETAILED DESCRIPTION OF THE PREFERRED EMBODIMENT

The present invention is further described in details with the accompanying figures and embodiments.

### Embodiment 1

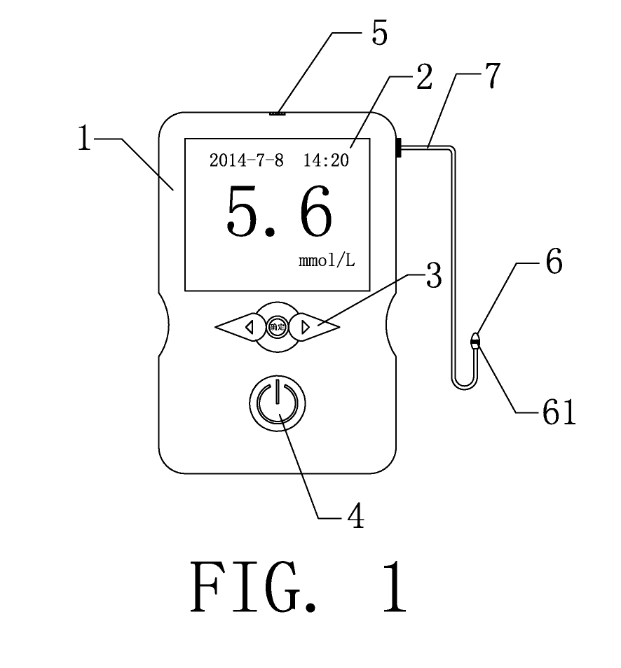

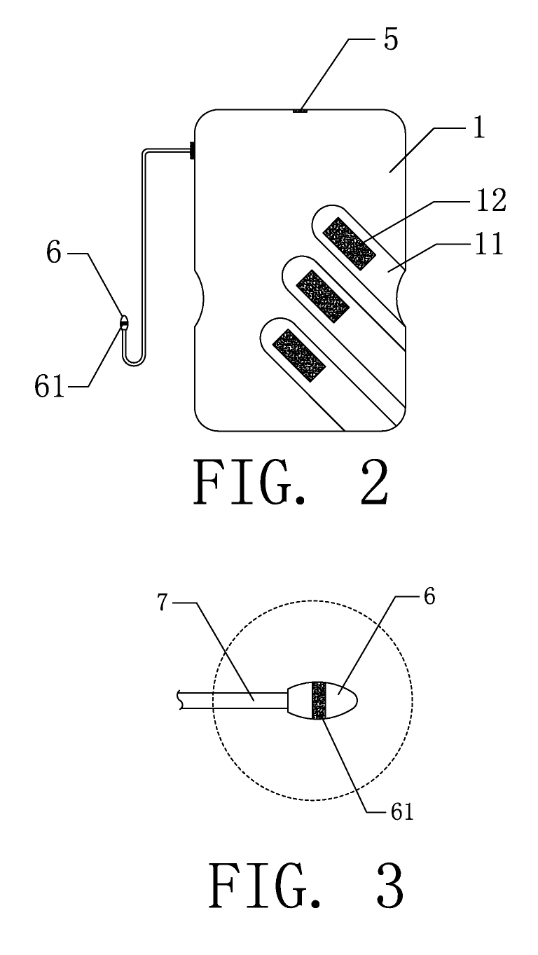

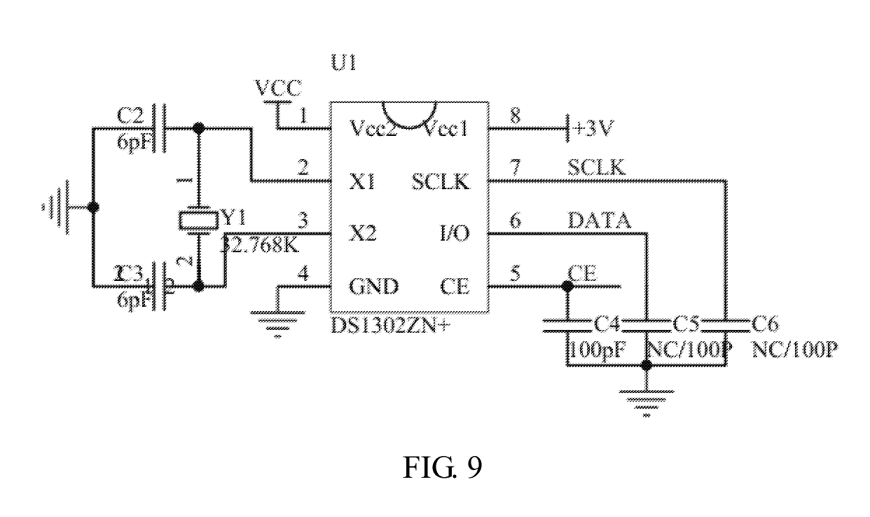

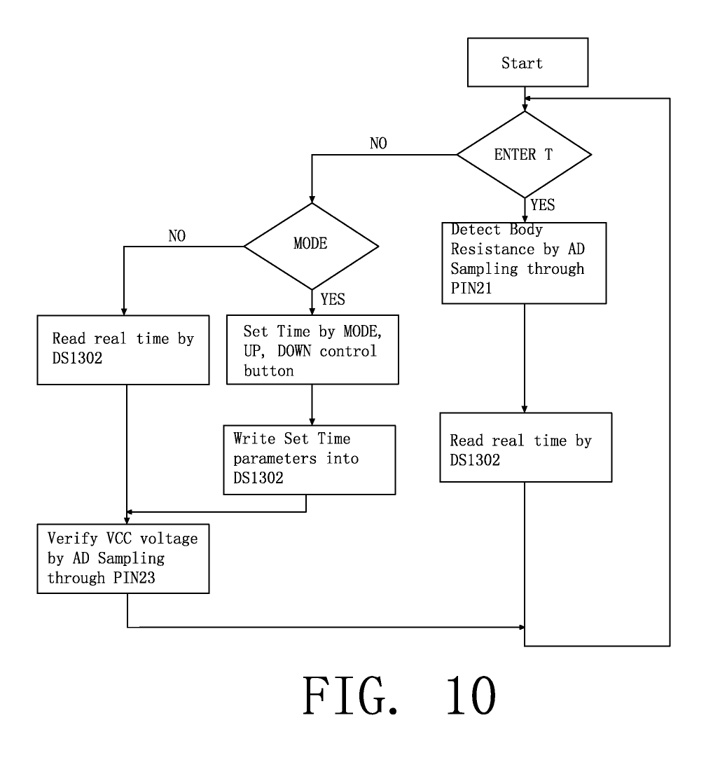

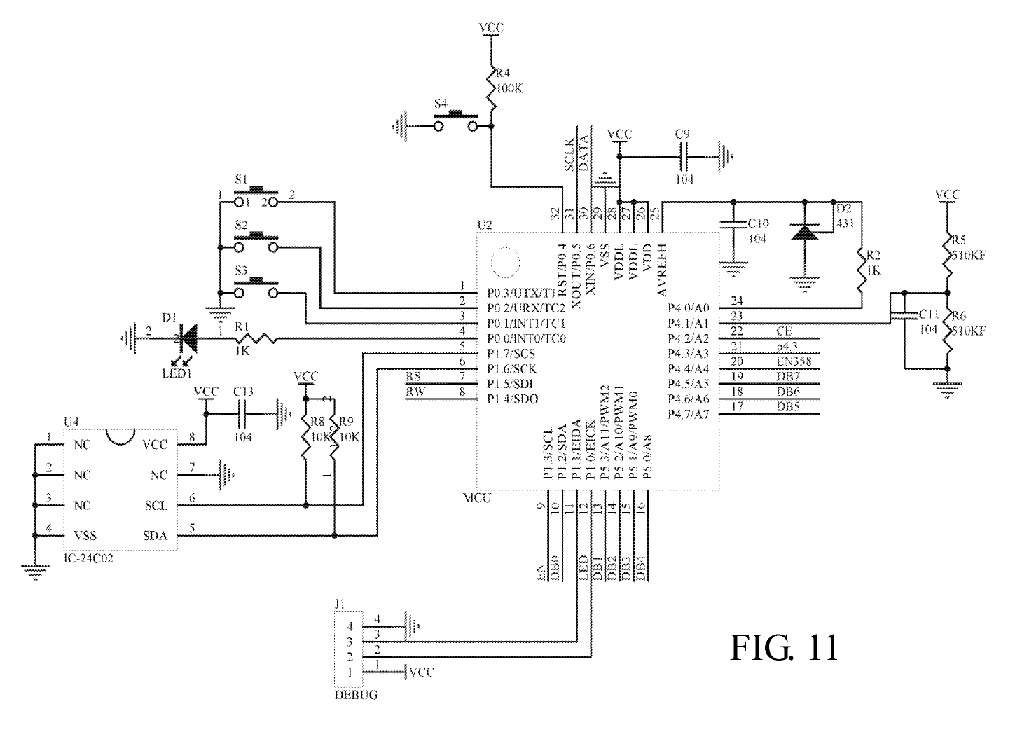

Referring to FIGS. 1 and 2 of the drawings, a bloodless glucose measuring and monitoring device comprises: a main computer 1 and a PCB provided inside the main computer 1. The PCB comprises a LCD display 2, a microprocessor, a blood glucose detection circuit electrically connected to the microprocessor, and a memory module, a power module and a timekeeping module electrically connected to the microprocessor. The blood glucose detection circuit comprises a blood glucose detection module. As shown in FIG. 9 of the drawings, the timekeeping module utilizes a timekeeping chip model DS1302ZN+. As shown in FIG. 11 of the drawings, the microprocessor utilizes chip model MSP430FG4617.

On the PCB, a body resistance acquisition circuit which is electrically connected to the microprocessor is provided. The body resistance acquisition circuit comprises a body resistance acquisition module which detects a body resistance between two points of skin of a human body and communicates with the microprocessor such that the body resistance obtained by the body resistance acquisition module is translated to a value of blood glucose level through the microprocessor and then the value of blood glucose level is displayed on the LCD display 2 of the main computer 1.

Referring to FIGS. 2 to 4 of the drawings, the main computer 1 comprises a main casing defining a bottom casing member and a plurality of positioning grooves 11 for guiding a position of a human finger provided on the bottom casing member of the main casing of the main computer 1. Each of the positioning grooves has a touch type metal electrode 12 provided on a front end of the corresponding positioning groove and electrically connected to an anode of the body resistance acquisition circuit. Preferably, each of the positioning grooves 11 has a finger-shaped structure. In particular, three positioning grooves 11 are provided, which are defined as an index finger groove, a middle finger groove and a ring finger groove respectively. When a bare index finger, a bare middle finger and a bare ring finger of a left hand of the human body are placed on the positioning grooves to press against the positioning grooves, each of the bare index, middle and ring fingers is in direct contact with the metal electrode portion of the corresponding positioning groove. Then a right hand of the human body holds the handheld electrode 6 such that the right hand is in direct contact with the metal electrode portion 61. Accordingly, a loop resistance is formed. The position of each of the positioning grooves 11 are arranged at a position which is convenience to the user to gripping the bloodless glucose monitoring device. In particular, the index, middle and ring finger of the left hand of the human body are pressing onto the positioning grooves 11, the thumb of the left hand of the human body is pressing onto a front surface of the main casing of the main computer 1 such that the thumb can be used to control the control button 3 and the on/off switch 4 provided on the front surface of the main casing of the main computer 1, and the small finger of the left hand can press against a bottom surface of the main casing of the main computer for enhancing the gripping position of the left hand. In order to provide a better contact surface for the fingers, the touch type metal electrode is provided on each of the positioning grooves such that the fingers can have a better contact point through the touch type metal electrode. The polarity of the touch type metal electrode 12 can be positive or negative. If the touch type metal electrode 12 is an anode, then the handheld electrode 6 is a cathode. The bloodless glucose monitoring device further comprises a connecting wire 6 having two proximal ends, namely a first end and a second end. The first end of the connecting wire is connected to a cathode of the body resistance acquisition circuit and extending from a side portion of the main casing and the second end of the connecting wire is connected to the handheld electrode 6. The handheld electrode 6 has a metal electrode portion exposing on an exterior surface of said handheld electrode connecting to metal wires of the connecting wire 7. Accordingly, the two hands of the user can control the bloodless glucose monitoring device of the present invention easily.

### Embodiment 2

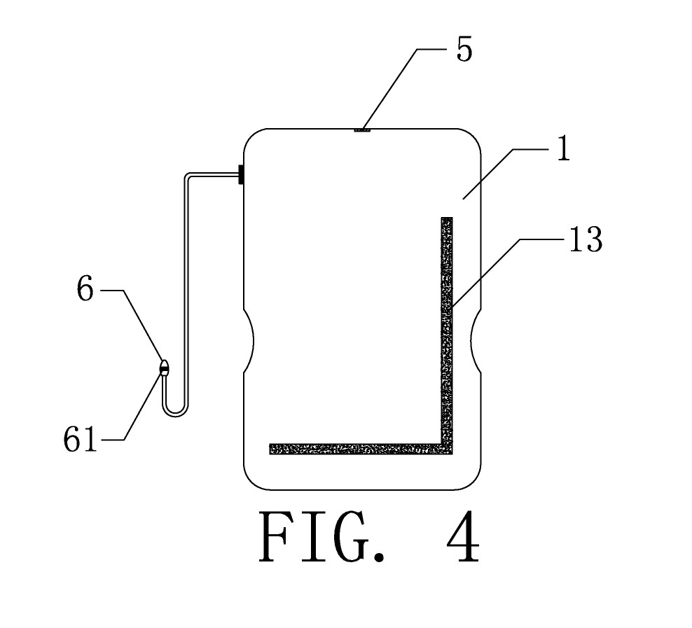

Referring to FIG. 4 of the drawings, a groove-less design can be used for the bottom surface of bottom casing member of the main computer 1. A touch type metal electrode 13 is provided on the bottom surface of the bottom casing member of the main computer 1 which covers at least a portion of the bottom surface of the bottom casing member of the main computer 1 in such a manner that the resistance generated from this touch type metal electrode 13 is insignificant to avoid any significant inaccuracy during using the bloodless glucose measuring and monitoring device of the present invention.

### Embodiment 3

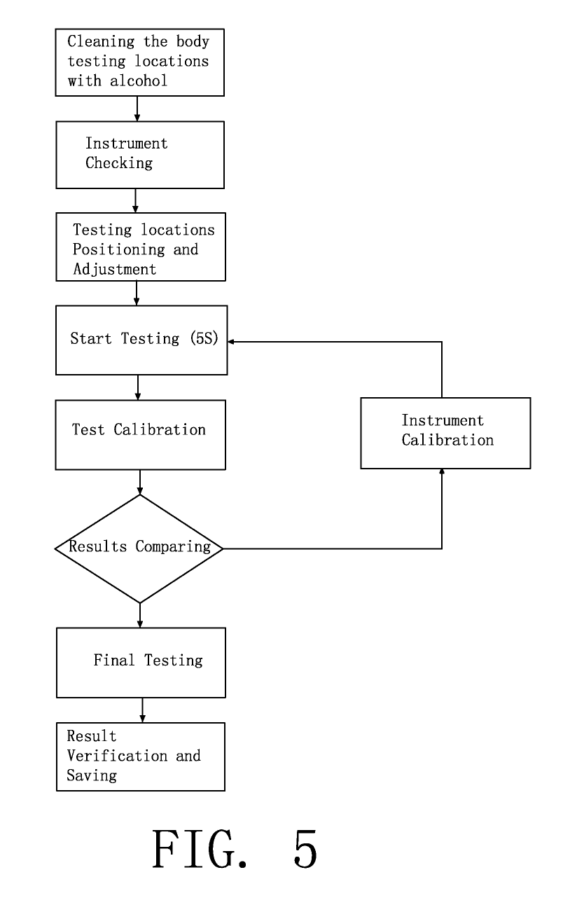

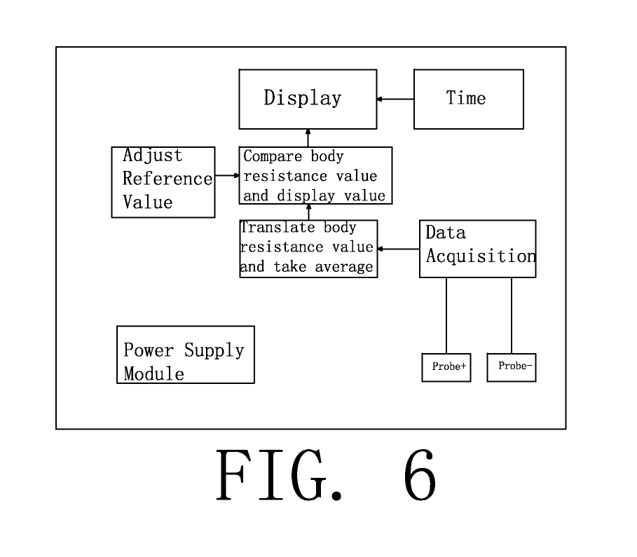

Referring to FIGS. 5 to 8 and FIG. 10 of the drawings, a method of use of the bloodless glucose measuring and monitoring device comprises the following steps:

(a) Select a plurality of locations of the human body, each of which is capable of reflecting a blood glucose level which is closely correlated with a body resistance of the particular location of the human body, wherein a change in body resistance of the particular location of the human body is directly correlated to a change in blood glucose level of the particular location of the human body and an amplitude of the change in blood glucose level and an amplitude of the change in body resistance resulted from the change in blood glucose level are the same. After the locations of the human body of which the body resistance and the blood glucose level are closely correlated are selected, such as left hand and right hand, it is determined that the index, middle and ring fingers of the left hand as well as the entire palm of the right hand are served as the contact points for resistance measurement based on the above description of the bloodless glucose monitoring device of the present invention. The sweat gland secretion of hands from different users are different, which will affect the contact effect between the hands and the metal and hence the accuracy of the test result. Accordingly, before testing, alcohol is required for cleaning the body parts for testing. The particular location of the human body selected for testing will directly affect the accuracy of the glucose test. In particular, body location which has a thinner skin at which the skin and the vein are closely positioned is preferred. The glucose content in a vein will have a direct effect on the chemical content of the skin at the particular location, while the skin resistance and the chemical content are closely correlated, and changes in blood glucose level at that particular position will also have direct impact on the skin resistance.

(b) Blood sampling is required for the first time for the present invention. Conduct a blood glucose test for obtaining a value of blood glucose level which is define as ‘blood glucose level a’ through the main computer. The main computer 1 has a test strip slot 5 provided at an upper portion of the main computer 1 and the blood glucose test is conducted through blood sampling by a test strip and inserting the test strip into the test strip slot 5 to start the bloodless glucose monitoring device and to obtain the blood glucose level a through the bloodless glucose monitoring device blood. For example, the value of ‘blood glucose level a’ is 5.5 mmol/L. In general, this blood glucose test need to be carried out under a healthy body condition. In addition, the values for before meal condition and after meal condition are required separately. Under the after meal condition, food are consumed by the user and the glucose content in the food will affect the blood glucose concentration in the bloodstream. This blood glucose test provides a standard value which is used for comparing the future test values to determine whether the user is under a healthy body condition.

(c) Obtain an average value of body resistance b through measuring a value of body resistance between two arms through the positioning grooves and the handheld electrode repeatedly for a predetermined number of times, therefore a correlation between the blood glucose level and the resistance under a particular body condition is obtained and defined as t=a/b. Body resistance mainly includes skin resistance. The resistance of stratum corneum of 0.05˜0.2 mm in epidermis is very great and is around 6˜10 kΩ or even reach 100 kΩ when the skin is dry. However, the stratum corneum is easily destroyed. The skin resistance is about 800˜1200 kΩ if the stratum corneum is removed. Accordingly, it is very important to use alcohol for cleaning in step (a) above. Assume the average resistance between two arms is 5 kΩ, then t=a/b=5.5 mmol/L/5 kΩ=1.1 mmol/L·kΩ.

(d) For each subsequent body resistance cn of selected body location, if the value is 5.5 kΩ, the corresponding blood glucose level value an can be calculated through the correlation t. The formula for calculation is: an=cn*t=5.5 kΩ*1.1 mmol/L·kΩ=6.05 mmol/L. The body resistance measured by the body resistance acquisition module is automatically processed through the microprocessor to translate into a blood glucose level, which is displayed on the LCD display 2 for the user's reference. Because the accuracy of display digit of the LCD display 2 is 0.1, the value displayed on the LCD display will be 6.1 mmol/L, that this value 6.1 mmol/L is greater than the standard value of 5.5 mmol/L by 0.6 mmol/L. Therefore, when the user has this data, he or she will need to pay special attention on his or her diet in order to maintain a level which is close to the standard value.

(e) Because the blood glucose level under before meal condition and after meal condition are different, a standard value for before meal condition and for after meal condition are required separately when the body is under a healthy condition. A blood glucose test with blood sampling for obtaining the standard value for before meal condition, which is define as ‘f’, and for after meal condition, which is defined as ‘g’, are required respectively. The values of ‘f’ and ‘g’ are obtained through blood glucose test with blood sample and these two values are used as a standard for subsequent blood glucose test.

(f) For each subsequent test at a particular time under the before meal condition, such as 12:00 pm, the value of blood glucose level under the particular condition and is obtained through the bloodless glucose monitoring device of the present invention. Then, and is compared with the standard value f such that the amplitude of blood glucose changes can be obtained, which can be used to remind the user about the blood glucose level changes on a daily basis such that the user can timely adjust his or her lifestyle.

(g) For each subsequent test at a particular time under the after meal condition, such as 16:00, the value of blood glucose level under the particular condition an2 is obtained through the bloodless glucose monitoring device of the present invention. Then, compare with the standard value g such that the amplitude of blood glucose changes can be obtained, which can be used to remind the user about the blood glucose level changes on a daily basis such that the user can timely adjust his or her lifestyle.

(h) The memory module automatically saves the value of blood glucose test and the user can timely adjust his or her lifestyle based on the changes in his or her blood glucose level.

The following table can be used to illustrate the blood glucose level changes in one week under a before meal condition of a particular user:

| | Monday | Tuesday | Wednesday | Thursday | Friday | Saturday | Sunday |
|---|---|---|---|---|---|---|---|
| Standard | 5.1 | 5.1 | 5.1 | 5.1 | 5.1 | 5.1 | 5.1 |
| value | | | | | | | |
| Before | 5.2 | 5.4 | 5.5 | 5.6 | 5.4 | 5.2 | 5.0 |
| meal, eg. | | | | | | | |
| 12:00pm | | | | | | | |
| Increased or | Increased | Increased | Increased | Increased | Increased | Increased | Decreased |
| decreased | | | | | | | |

From the above table which shows the test results under a before meal condition, the blood glucose level is increased continuously from Monday through Thursday. This kind of increase is not a good phenomenon. Through the level is decreased on Friday, the level is still higher than the standard value. Therefore, diet control is still required for maintaining a good eating habit.

The following table can be used to illustrate the blood glucose level changes in one week under an after meal condition of a particular user:

| | Monday | Tuesday | Wednesday | Thursday | Friday | Saturday | Sunday |
|---|---|---|---|---|---|---|---|
| Standard | 5.5 | 5.5 | 5.5 | 5.5 | 5.5 | 5.5 | 5.5 |
| value | | | | | | | |
| Before | 5.7 | 5.5 | 5.6 | 5.7 | 5.4 | 5.9 | 6.0 |
| meal, eg. | | | | | | | |
| 12:00pm | | | | | | | |
| Increased or | Increased | Leveled | Increased | Increased | Decreased | Increased | Increased |
| decreased | | | | | | | |

The standard value under an after meal condition is usually relatively higher. When the test value is decreased, or when the decreased level is not great, the user does not need to have any particular adjustment and can continue his or her normal diet. However, when the blood glucose level is increased, appropriate adjustment to diet is required. The tests under a before meal condition and the test under an after meal condition are similar. Through the data of blood glucose level obtained on a daily basis, the blood glucose level can be monitored. Unlike using conventional glucometers which required blood sampling for testing, the device of the present invention can greatly reduce the time for testing and lower the pain level for testing.

In addition, the blood glucose monitoring for user with elevated blood glucose level or for diabetes patient are illustrated as follows:

Table for user with elevated blood glucose

| | Monday | Tuesday | Wednesday | Thursday | Friday | Saturday | Sunday |
|---|---|---|---|---|---|---|---|
| Standard | 6.5 | 6.5 | 6.5 | 6.5 | 6.5 | 6.5 | 6.5 |
| value | | | | | | | |
| Before | 6.6 | 6.7 | 6.8 | 6.9 | 6.8 | 6.6 | 6.5 |
| meal, eg. | | | | | | | |
| 12:00pm | | | | | | | |
| Increased or | Increased | Increased | Increased | Increased | Increased | Increased | Leveled |
| decreased | | | | | | | |

From the above table, the standard values of blood glucose level are between 6.1˜6.9, which belongs to the zone with high probability of developing diabetes and is classified as a sub-health state. Accordingly, if the blood glucose level is increased continuously, a diet change is required or a visit to hospital for diagnosis is needed to prevent the development of diabetes. In this aspect, the monitoring effect provided by the bloodless glucose monitoring device of the present invention is very great.

Table for diabetes patients

| | Monday | Tuesday | Wednesday | Thursday | Friday | Saturday | Sunday |
|---|---|---|---|---|---|---|---|
| Standard | 7.2 | 7.2 | 7.2 | 7.2 | 7.2 | 7.2 | 7.2 |
| value | | | | | | | |
| Before | 7.3 | 7.4 | 7.5 | 7.6 | 7.5 | 7.4 | 7.3 |
| meal, eg. | | | | | | | |
| 12:00pm | | | | | | | |
| Increased or | Increased | Increased | Increased | Increased | Increased | Increased | Increased |
| decreased | | | | | | | |

The above table shows the results of user with diabetes. From the above table, the glucose levels are higher than the standard values, which illustrates that he blood glucose is increased continuously. Accordingly, the patient should go to visit a doctor as soon as possible so as to prevent any life-threatening conditions. In this aspect, the bloodless glucose monitoring device of the present invention can be used as preventive measures for diabetes.

In the step (d) above, the measurement of body resistance by the body resistance acquisition module are carried out through two steps. The first step includes verifying a Vcc voltage. As shown in FIG. 11, the pin number 23 is sampled for 34 times, the greatest and the smallest values are removed to obtain an average AD value through the following formula:

$$Vcc = \frac{\frac{AD ◯ 1}{256} \times 2.5 \times ( 510 k + 510 k )}{510 k} = \frac{AD / D \times 5}{256}$$

The second step includes measuring a body resistance. As shown in FIG. 11, the pin number 21 is sampled for each 4 ms for 1000 times, the time used is 4 s, the AD value is obtained after taking average through the following formula:

$$\frac{Vcc \cdot \frac{10 k}{10 k + {R}_{body}} \times 10}{2.5} \times 256 = {AD}_{◯ 2}$$

Accordingly, the body resistance is obtained through the following formula:

$${R}_{body} = \frac{1024 \times Vcc}{{AD}_{◯ 2}} - 10 k ;$$

where R_{body} refers to the value of body resistance. According to this formula, Vcc is constant in general while the value of R_{body} is decreased as the value of AD is increased. As the blood glucose value an=cn*t, where cn refers to the value of R_{body} in the above formula, therefore the blood glucose value an will increase with an increase in R_{body}.

### Embodiment 4

A method of selecting a location of the human body which is capable of reflecting a blood glucose level of the human body. The location of the human body has direct effect on the accuracy of the blood glucose value measured by the bloodless glucose monitoring device according to the preferred embodiment of the present invention. The location being selected has a relatively thin skin layer is selected. This particular location is in close proximity to a vein of the human body (including the capillaries), that a blood glucose content in the vein has direct effect on a chemical composition of the skin which is in close proximity to the vein while a skin resistance is closely correlated to the chemical composition of the skin. Accordingly, changes in blood glucose level will have a direct effect on the skin resistance at the selected location. For example, the skin on the first segment of left index finger near to the right upper side, the skin on the first segment of left index finger near to the right lower side and the skin on the first segment of ring finger near the left side are belonged to locations of human body of which the skin is in close proximity to a vein (capillaries). Before the bloodless glucose test, pre-test treatment includes: sterilizing the skin of selected location with alcohol for more than 30 seconds such that the skin of selected location can reach a stabilized environment conditions, including stable temperature, humidity and salinity, for testing. In addition, a mid-frequency wave can be used to stimulate the skin to obtain a normal impedance reaction.

### Embodiment 5

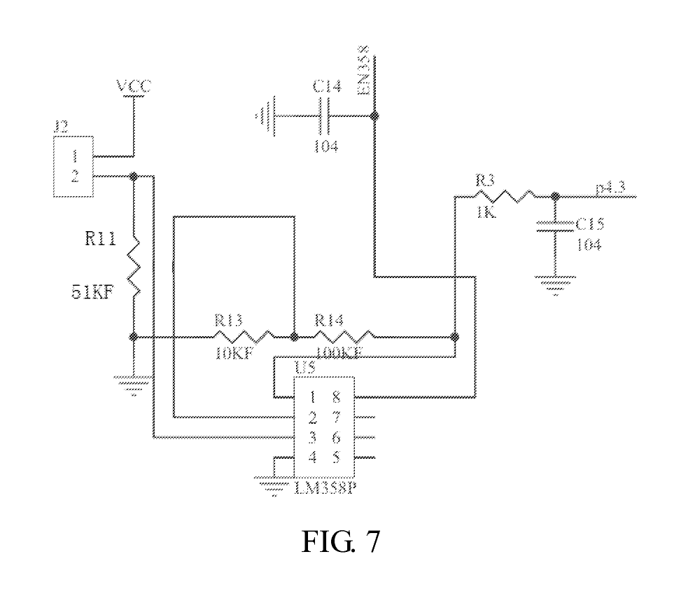

Referring to FIG. 7 of the drawings, which illustrates a circuit diagram of a body resistance acquisition circuit of the bloodless glucose measuring and monitoring device according to the above preferred embodiment of the present invention. In the circuit diagram, a dual operational amplifier, model LM358P, is used which is capable of providing high stability while the cost is low. This chip is formed by two independent, high-gain frequency-compensated operational amplifiers which can operate over a power source voltage from 3V through 32V from a single supply or operate independently from two supply condition. Also, this amplifier has internal frequency compensation. The pin number 3 of the LM358P is connected to the trigger J1, the pin number 1 is sequentially connected to a resistant R3 (1K) and capacitance C15-104. The pin number 8 is connected to capacitance C14-104. The pin number 3 is connected to R13-51KF. The R13-51KF is grounded. The terminal of R13-51KF which is connected to the ground and the connecting terminal of the pin number 1 are connected to R13-10KF and R14-100KF in series. The pin number 2 is connected between R13-10KF and R14-100KF.

### Embodiment 6

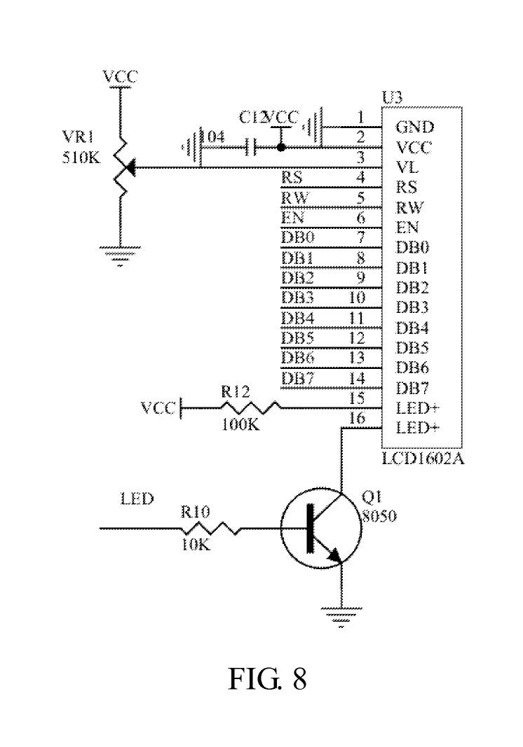

Referring to FIG. 8 of the drawings, a circuit diagram of a display module of the LCD display of the bloodless glucose measuring and monitoring device according to the above preferred embodiment of the present invention is illustrated. In the circuit, a display, model number LCD1602A, is connected. The pin number 3 of the LCD1602A display is connected to variable resistance VR1-510K, the pin number 15 is connected to resistance R12-100K, the pin number 16 is connected to a transistor Q1-8050, and the negative terminal of the transistor Q1-8050 is connected to the resistance R10-10K.

### Embodiment 7

Referring to FIG. 9 of the drawings, a circuit diagram of a timekeeping module of the bloodless glucose measuring and monitoring device according to the above preferred embodiment of the present invention is illustrated. In the circuit of the timekeeping module, a timekeeping chip DS1302ZN+ is connected, which is a real-time timekeeping chip with high efficiency, low power consumption and internal RAM. This timekeeping chip can keep time based on year, month, day, day of the week, hour, minute and second, and provide leap year compensation. The working voltage is 2.5V˜5.5V. A ‘three-wire interface’ is used to connect to CPU for synchronous communication while multiple byte data transfer for clock information or RAM data can be transmitted through burst mode. DS1302 has 31×8 RAM internally for temporary data storage. DS1302 is an upgraded version of DS1202 and is compatible with DS1202. In DS1302, additional features of dual-power pins for primary and back-up power supplies and trickle charger function for back-up power supplies are provided.

### Embodiment 8

Referring to FIG. 11 of the drawings, a circuit diagram of a microprocessor of the bloodless glucose monitoring device according to the above preferred embodiment of the present invention is illustrated. The chip model of the microprocessor is MSP430FG4617 of which pins 1, 2 and 3 are connected to three control buttons 3 at a front side of the main computer 1 below the LCD display respectively. Pin 32 is connected to on/off switch 4. Pins 5 and 6 are connected to memory chip, that the chip model is IC-24C02. Pin 4 is sequentially connected to resistance R1-1K, photodiode Dl respectively. The other terminal of the photodiode Dl is connected to ground. Pin 23 is connected to voltage dividing resistor R5-510KF and R6-510KF. The capacitance C11-104 and the resistance R6-510KF are connected in parallel. The body resistance acquisition circuit can calculate the resistance between two points of the human body through this voltage dividing resistance.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A bloodless glucose monitoring device and a method of use thereof, said bloodless glucose monitoring device comprises: a main computer (1) and a PCB provided in said main computer (1), wherein said PCB comprises a LCD display (2); a microprocessor; a blood glucose detection circuit connected to said microprocessor which comprises a blood glucose detection module; and a memory module, a power module and a timekeeping module connected to said microprocessor, characterized in that: said PCB comprises a body resistance acquisition circuit electrically connected to said microprocessor, wherein said body resistance acquisition circuit comprises a body resistance acquisition module which detects a body resistance between two points of skin of a human body and communicates with said microprocessor such that the body resistance obtained by said body resistance acquisition module is translated into a blood glucose value through said microprocessor and then the blood glucose value is displayed through said LCD display (2) of said main computer (1).

2. The bloodless glucose monitoring device and a method of use thereof according to claim 1, characterized in that, said main computer (1) comprises a main casing defining a bottom casing member; at least two positioning groves (11) for guiding a position of a human finger; and a touch type metal electrode (12) provided on a front end of each said positioning groove (11) connecting to an anode of said body resistance acquisition circuit.

3. The bloodless glucose monitoring device and a method of use thereof according to claim 1, characterized in that, said device comprises a connecting wire (7) having a first end and a second end; and a handheld electrode (6) having a metal electrode portion (61) peripherally provided on an exterior surface of said handheld electrode (6), wherein said first end of said connecting wire (7) is connected to a cathode of said body resistance acquisition circuit and extending from a side portion of said main casing and said second end of said connecting wire (7) is connected to said metal electrode portion (61) of said handheld electrode (6).

4. The bloodless glucose monitoring device and a method of use thereof according to claim 2, characterized in that, said device comprises three said positioning grooves (11) defining an index finger groove, a middle finger groove and a ring finger groove arranged for placing a bare index finger, a bare middle finger and a bare ring finger of a human body onto said three positioning grooves (11) respectively in such a manner that each of the bare index, middle and ring fingers of a left hand of the human body are having a direct contact with said touch type metal electrode inside said corresponding positioning groove (11) through pressing against said corresponding positioning groove while a bare right hand of the human body is in direct contact with said metal electrode portion (61) through holding said handheld electrode (6), thereby a loop resistance is formed.

5. The bloodless glucose monitoring device and a method of use thereof according to claim 1, characterized in that, said device comprises a dual operational amplifier connecting to said body resistance acquisition circuit.

6. The bloodless glucose monitoring device and a method of use thereof according to claim 1, characterized in that, said method of use of said device comprises the steps of:
(a) selecting a plurality of locations of the human body, each of which is capable of reflecting a blood glucose level of the human body of which the blood glucose level is closely correlated with a body resistance of the particular location of the human body, wherein a change in body resistance of the particular location of the human body is directly correlated to a change in blood glucose level of the particular location of the human body and an amplitude of the change in blood glucose level and an amplitude of the change in body resistance resulted from the change in blood glucose level are the same;
(b) conducting a blood glucose test with blood sample for obtaining a value of blood glucose level which is define as ‘blood glucose value a’ through said main computer (1), wherein said main computer (1) has a test strip slot (5) provided at an upper portion of said main computer (1) and said blood glucose test is conducted through blood sampling by a test strip and inserting the test strip into the test strip slot (5) to start said bloodless glucose monitoring device and to obtain the blood glucose value a through said bloodless glucose monitoring device blood;
(c) obtaining an average value of body resistance b through measuring a value of body resistance between two arms through said positioning grooves and said handheld electrode repeatedly for a predetermined number of times, therefore a correlation between the blood glucose value and the body resistance under a particular body condition is obtained and defined as t=a/b;
(d) after the correlation t=a/b is established, carrying out a bloodless blood glucose test by obtaining a blood glucose value ‘an’ through measuring a body resistance ‘cn’ of a particular body location and utilizing the correlation t for calculation, where an=cn*t, wherein the body resistance measured by said body resistance acquisition module is automatically processed through said microprocessor to translate into the blood glucose value, which is displayed through said LCD display (2);
(e) determining a value of blood glucose level standard under a particular time condition since a value of blood glucose level at different times will be different, through conducting a blood glucose test with blood sample through said main computer under a healthy body condition of the human body, measuring the value of glucose level standard which is defined as standard glucose value f, where f is obtained through conducting the blood glucose test with blood sample and is used as a standard for subsequent bloodless blood glucose test to observe the changes in blood glucose level;
(f) subsequently obtaining a blood glucose value an1 under the particular time condition in the step (e) by carrying out the bloodless blood glucose test with said bloodless glucose monitoring device; comparing the blood glucose value an1 and the standard glucose value f to obtain an amplitude of change in blood glucose level such that the user can be reminded of the changes on a daily basis and react to the changes on a timely basis; and
(g) saving the blood glucose value for each of the blood glucose test carried out through said bloodless glucose monitoring device by said memory module such that the user can take appropriate measures in response to changes in blood glucose level.

7. The bloodless glucose monitoring device and a method of use thereof according to claim 6, characterized in that, in the step (a) of the method of use of said device, the locations of the human body has direct effect on the accuracy of the blood glucose value measured by said bloodless glucose monitoring device, wherein the locations being selected has relatively thin skin layer and is in close proximity to a vein of the human body, wherein a blood glucose content in the vein has direct effect on a chemical composition of the skin which is in close proximity to the vein while a skin resistance is closely correlated to the chemical composition of the skin such that a blood glucose level change has direct effect on the skin resistance at the locations being selected.

8. The bloodless glucose monitoring device and a method of use thereof according to claim 6, characterized in that, in the step (d) of the method of use of said device, the body resistance measured by the body resistance acquisition module comprises a measurement process comprising two steps, which is a first step and a second step, wherein the first step refers to verifying a Vcc voltage through resistance sampling for 34 times, removing the smallest value an1 the greatest value to obtain an average value AD through formula (a):
$$Vcc = \frac{\frac{AD ◯ 1}{256} \times 2.5 \times ( 510 k + 510 k )}{510 k} = \frac{AD / D \times 5}{256}$$
wherein the second step refers to measuring a body resistance through resistance sampling for every 4 ms for 1000 times, where a total time used is 4 s and an average AD value is obtained through formula (b):
$$\frac{Vcc \cdot \frac{10 k}{10 k + {R}_{body}} \times 10}{2.5} \times 256 = {AD}_{◯ 2}$$
thereby the body resistance of human body is obtained through formula (c):
$${R}_{body} = \frac{1024 \times Vcc}{{AD}_{◯ 2}} - 10 k ;$$
where the R_{body} refers to the body resistance of human body as measured.
